(19) United States
(12) Patent Application Publication
(10) Pub. No.: US 2022/0181011 A1
(43) Pub. Date: Jun. 9, 2022

(54) SYSTEM, MEDICAL DEVICE, COMPUTER, AND MANAGING METHOD

(71) Applicant: NIHON KOHDEN CORPORATION, Tokyo (JP)

(72) Inventors: Takayuki SUGIYAMA, Tokorozawa-shi (JP); Fumito HORIUCHI, Tokorozawa-shi (JP); Yoshio SAKAI, Tokorozawa-shi (JP)

(73) Assignee: NIHON KOHDEN CORPORATION, Tokyo (JP)

(21) Appl. No.: 17/537,459

(22) Filed: Nov. 29, 2021

(30) Foreign Application Priority Data
Dec. 7, 2020 (JP) 2020-202739

(51) Int. Cl. G16H 40/40; A61N 1/04; G06K 7/14

(52) U.S. Cl. CPC G16H 40/40; G06K 7/1417; A61N 1/046; A61N 1/0492

(57) ABSTRACT

A system includes a medical device that uses a plurality of expendables each having an expiration date, and a computer that can be connected for communication to the medical device. The medical device includes an acquisition unit configured to acquire expiration information relating to an expiration date of a first expendable included in the plurality of expendables in a state that the medical device is not connected to the first expendable, and a transmission unit configured to transmit the expiration information acquired by the acquisition unit. The computer includes a reception unit configured to receive the expiration information, and a managing unit configured to manage the expiration date of the first expendable on the basis of the expiration information received by the reception unit.

### CROSS-REFERENCE TO RELATED APPLICATIONS

The present application claims priority from Japanese Patent Application No. 2020-202739, filed on Dec. 7, 2020, the entire content of which is incorporated herein by reference.

### TECHNICAL FIELD

The present invention relates to a system, a medical device, a computer, and a managing method.

### BACKGROUND ART

JP-A-2011-175566 discloses a system that manages the state of an automated external defibrillator (also referred to as an “AED” below) remotely by checking the expiration date etc. of disposable electrode pads connected to it and transmitting information obtained on the basis of check results to another device.

Incidentally, among methods for acquiring the expiration date of disposable electrode pads is a method in which a connector of the electrode pads holds information relating to the expiration date and an AED main body acquires that information from the connector when the connector is connected to the AED main body (disclosed in JP-A-2010-63771, for example).

Although the system disclosed in JP-A-2011-175566 enables remote management of the expiration date etc. of electrode pads of an AED, it cannot be applied to semiautomated defibrillators installed in hospitals etc. Since an AED is usually in a state that the electrode pads are connected to the AED main body, it is possible to acquire information relating to their expiration date from the AED at desired timing. However, since a semiautomated defibrillator is usually in such a state as not to be connected to the electrode pads and is not connected to the electrode pads until defibrillation is performed, a system as disclosed in JP-A-2011-175566 cannot manage the expiration date etc. of the electrode pads of the semiautomated defibrillator. This problem is not limited to semiautomated defibrillators but common to medical devices that uses expendables.

### SUMMARY

An object of the present disclosure is to enable, in a medical device using an expendable having an expiration date as in semiautomated defibrillators, remote management of the expiration date of an expendable even if the expendable is not connected to the medical device.

A system according to a first aspect of the disclosure is a system that includes a medical device that uses a plurality of expendables each having an expiration date and a computer that can be connected for communication to the medical device, wherein:

the medical device includes:
an acquisition unit configured to acquire expiration information relating to an expiration date of a first expendable included in the plurality of expendables in a state that the medical device is not connected to the first expendable; and
a transmission unit configured to transmit the expiration information acquired by the acquisition unit; and

the computer includes:
a reception unit configured to receive the expiration information; and
a managing unit configured to manage the expiration date of the first expendable on the basis of the expiration information received by the reception unit.

A medical device according to a first aspect of the disclosure is a medical device that uses a plurality of expendables each having an expiration date and can be connected for communication to a computer, comprising:

an acquisition unit configured to acquire expiration information relating to an expiration date of a first expendable included in the plurality of expendables in a state that the medical device is not connected to the first expendable; and

a transmission unit configured to transmit the expiration information acquired by the acquisition unit to the computer.

A computer according to a third aspect of the disclosure is a computer that can be connected for communication to a medical device that uses a plurality of expendables each having an expiration date, wherein:

the medical device acquires expiration information relating to an expiration date of a first expendable included in the plurality of expendables in a state that the medical device is not connected to the first expendable and transmits the acquired expiration information to the computer; and

the computer comprises:
a reception unit configured to receive the expiration information; and
a managing unit configured to manage the expiration date of the first expendable on the basis of the expiration information received by the reception unit.

A managing method according to a fourth aspect of the disclosure is an expendable managing method that is performed in a system including a medical device that uses a plurality of expendables each having an expiration date and a computer that can be connected for communication to the medical device, comprising:

the steps, executed in the medical device, of:
acquiring expiration information relating to an expiration date of a first expendable included in the plurality of expendables in a state that the medical device is not connected to the first expendable; and
transmitting the acquired expiration information; and

the steps, executed in the computer, of:
receiving the expiration information; and
managing the expiration date of the first expendable on the basis of the received expiration information.

### BRIEF DESCRIPTION OF DRAWINGS

FIG. 1 is a schematic diagram illustrating an example configuration of a system according to an embodiment of the present disclosure.

FIG. 2 is a block diagram illustrating an example configuration of each medical device according to the embodiment of the disclosure.

FIG. 3 is a schematic diagram illustrating an example expendable used in each medical device employed in the embodiment of the disclosure.

FIG. 4 is a block diagram illustrating an example configuration of a computer according to the embodiment of the disclosure.

FIG. 5 is a flowchart illustrating an example process that is executed by the system according to an embodiment of the disclosure.

FIG. 6 is a diagram illustrating a specific example of expiration date management according to the embodiment of the disclosure.

### DESCRIPTION OF EMBODIMENT

An embodiment of the present disclosure will be hereinafter described with reference to the drawings. In the following description, the same or equivalent elements shown in even different figures are given the same symbol or name and will not be described redundantly.

### (System Configuration)

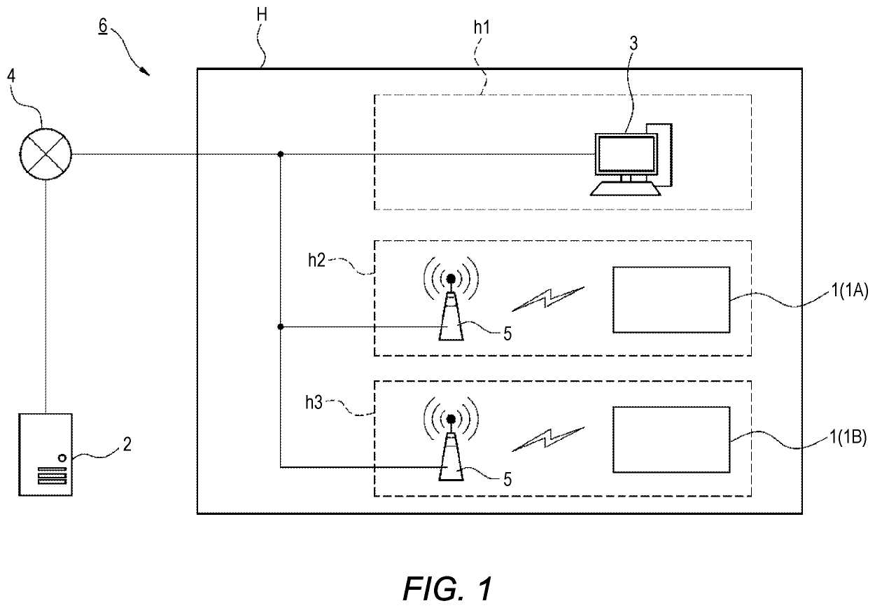

First, the configuration of a system according to the embodiment will be described using FIG. 1. FIG. 1 is a schematic diagram illustrating an example configuration of a system. The system 6 shown in FIG. 1 can include medical devices 1 (in this example, medical devices 1A and 1B), a computer 2, and a user terminal 3.

In the example of FIG. 1, the medical devices 1 and the user terminal 3 are installed in a medical institution H and the computer 2 is installed outside the medical institution H. The user terminal 3 may be disposed outside the medical institution H. The medical devices 1, the user terminal 3, and the computer 2 can be connected for communication to each other wirelessly or by wire via a communication network 4. The medical devices 1 and each of the computer 2 can be connected for communication to each other wirelessly or by wire via a LAN (local area network) or the like.

The medical devices 1 are devices that use an expendable having an expiration date. This specification is mainly directed to an example case that the medical devices 1 are semiautomated defibrillators, they may be medical devices other than semiautomated defibrillators. Semiautomated defibrillators are used in medical institutions, ambulances, etc. For example, a semiautomated defibrillator is provided with an auto mode in which the semiautomated defibrillator makes a judgment as to whether to deliver an electric shock, determination of electric shock energy, etc. and a manual mode in which a healthcare professional or the like makes a judgment as to whether to deliver an electric shock, determination of electric shock energy, etc. Although in this example the medical devices 1 are connected for communication via respective wireless routers 5, they may be connected for communication by wire.

The computer 2 is a server that manages, remotely, the expiration dates of the expendables used by the medical devices 1. The computer 2 may be a server that provides what is called a cloud-type service. It is possible to cause the computer 2 to also manage, remotely, the expiration dates of expendables used in a medical institution other than the medical institution H.

For example, the user terminal 3 is a terminal device that manages medical devices of the medical institution H. The user terminal 3 may be a server of a medical devices management system provided in the medical institution H. In the case of this configuration, the user terminal 3 can be connected for communication to other user terminals (not shown) that function as client devices of the medical devices management system.

For example, the medical institution H is a medical care facility such as a hospital, a clinic, or a welfare nursing facility for the elderly. In this example, the medical institution H includes areas h1-h3. The area h1 is an area in which the user terminal 3 is installed and is, for example, an ME (medical engineer) room. Each of the areas h2 and h3 is an area in which the medical device 1 is installed and is, for example, a hospital ward, an intensive care room, a surgery room, or the like.

### (Medical Devices)

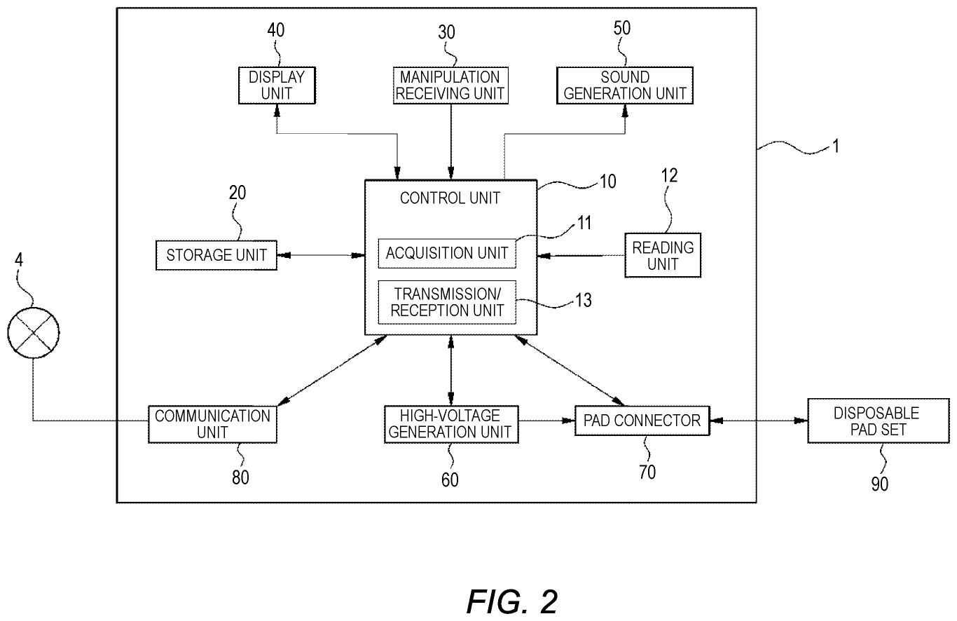

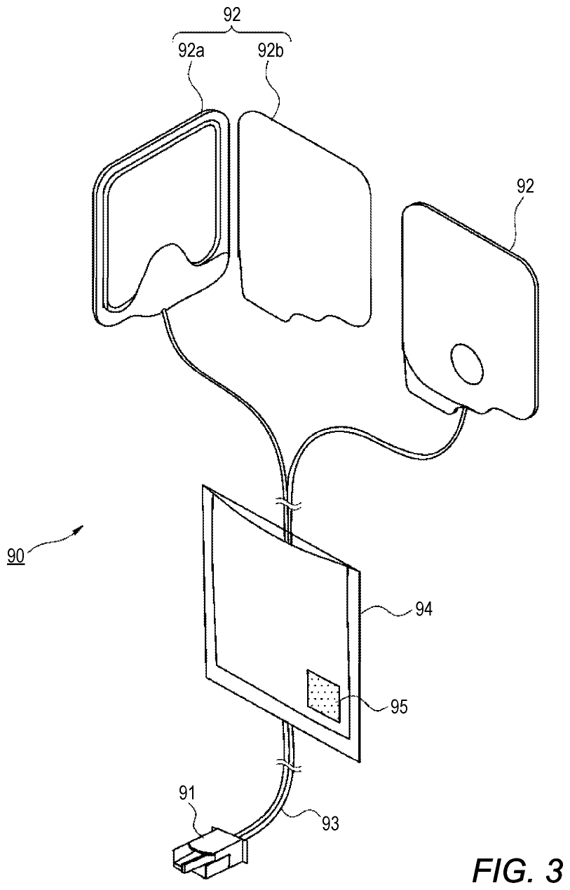

Next, the medical devices 1 will be described using FIGS. 2 and 3. FIG. 2 is a block diagram illustrating an example configuration of each medical device according to the embodiment. FIG. 3 is a schematic diagram illustrating an example expendable used by each medical device according to the embodiment.

The medical device 1 shown in FIG. 2 is a semiautomated defibrillator. In the following description, each medical device 1 may also be referred to as a “semiautomated defibrillator 1.” As show in FIG. 2, the semiautomated defibrillator 1 can include a control unit 10, a storage unit 20, a manipulation receiving unit 30, a display unit 40, a sound generation unit 50, a high-voltage generation unit 60, a pad connector 70, and a communication unit 80.

The semiautomated defibrillator 1 can be connected to a disposable pad set 90 having an expiration date. The disposable pad set 90, which is a pair of disposable electrode pads, is connected to the pad connector 70 when it is used. The semiautomated defibrillator 1 may further be equipped with external paddles or internal paddles in addition to the pad connector 70 to be connected to the disposable pad set 90.

As shown in FIG. 3, the disposable pad set 90 is equipped with a connector 91, two electrode pads 92, and cables 93.

The connector 91 is to be connected to pad connector 70. The connector 91 may be equipped with a holding unit (not shown) that holds expiration information that is information relating to an expiration date of the disposable pad set 90. The expiration information held by the holding unit is made readable by an acquisition unit 11 (described later) in a state the connector 91 is connected to the pad connector 70. The configuration of the holding unit may be a known one (there are no particular limitations on it). The holding unit may include a memory.

Each electrode pad 92 includes an attachment surface 92a which is provided with adhesive gel or a metal foil and a peelable sheet 92b. When the semiautomated defibrillator 1 is used, the peelable sheet 92b is peeled off and the attachment surface 92a is attached to the body of a patient. The cables 93 connect the connector 91 and the electrode pads 92 electrically.

Before being used, the disposable pad set 90 is stored in such a state as to be sealed in a wrapping member 94. The wrapping member 94 is an attachment to the disposable pad set 90 and is employed to prevent drying of the adhesive gel and for other purposes. In a state that the disposable pad set 90 is stored, the connector 91 and parts of the cables 93 may be exposed being located outside the wrapping member 94.

An identification code 95 is attached to the wrapping member 94. The identification code 95 includes the expiration information of the disposable pad set 90. The expiration information included in the identification code 95 can be read by an acquisition unit 11 (described later). There are no particular limitations on the identification code 95 except that it should be able to include expiration information; for example, it is a QR code (registered trademark) or a bar code.

There are no particular limitations on the location of the identification code 95 except that it should be located at such a position as to be able to be read in a state that the wrapping member 94 is not opened; the location of the identification code 95 may be either an outer surface of the wrapping member 94 or the main bodies or some other attachment to the disposable pad set 90. It suffices that the disposable pad set 90 and the attachment to it be provided with at least one of the holding unit and the identification code 95.

Returning to the description of FIG. 2, the control unit 10 controls various operations of the semiautomated defibrillator 1 by reading programs etc. stored in the storage unit 20 and running the programs. For example, the control unit 10 can include a processor such as a CPU (central processing unit), memories such as a ROM (read-only memory) and a RAM (random access memory), an A/D converter, etc.

The control unit 10 controls various operations for life saving such as a charging/discharging control, a sequence control, A/D conversion, and an analysis of an electrocardiogram. Furthermore, the control unit 10 functions as an acquisition unit 11 and a transmission/reception unit 13.

The acquisition unit 11 acquires expiration information of a first disposable pad set 90 in a state that the acquisition unit 11 is not connected. In the following, the configuration etc. of the acquisition unit 11 will be described for each of three cases, that is, a case that the connector 91 has the holding unit, a case that the wrapping member 94 has the identification code 95, and a case that the connector 91 has the holding unit and the wrapping member 94 has the identification code 95.

Where the connector 91 has the holding unit, the acquisition unit 11 reads expiration information of a second disposable pad set 90 from the holding member of the second disposable pad set 90 when the second disposable pad set 90 is connected to the semiautomated defibrillator 1. Then the acquisition unit 11 acquires the expiration information of the second disposable pad set 90 as expiration information of the first disposable pad set 90 to be connected to the semiautomated defibrillator 1 next.

In general, it is considered that disposable pad sets 90 are used in order of the length of the period to the expiration date (shortest first). That is, it is considered that a disposable pad set 90 to be used next has a period to an expiration date that is longer than or equal to that of a disposable pad set 90 that was used last time. Based on this notion, in acquiring expiration information from the holding unit of the connector 91, the acquisition unit 11 acquires the expiration information of the second disposable pad set 90 as expiration information of the first disposable pad set 90.

Where the wrapping member 94 has the identification code 95, the acquisition unit 11 may acquire, as expiration information, information obtained by reading the identification code 95 by the reading unit 12. That is, the acquisition unit 11 may acquire, as expiration information of the first disposable pad set 90 to be connected to the semiautomated defibrillator 1 next to the second disposable pad set 90, information obtained by reading the identification code 95 attached to the second disposable pad set 90 or the wrapping member 94 of the second disposable pad set 90. For example, the reading unit 12 is a code reader capable of reading an identification code by scanning it. Where the disposable pad set 90 does not have an identification code 95, the semiautomated defibrillator 1 need not be equipped with the reading unit 12.

Where the first disposable pad set 90 is located near the semiautomated defibrillator 1, the acquisition unit 11 may acquire, as expiration information of the first disposable pad set 90, the expiration information read from the identification code 95 of the first disposable pad set 90. In the case of this configuration, in response to reading of the identification code 95 of the first disposable pad set 90 by the acquisition unit 11 before connection of the first disposable pad set 90 to the semiautomated defibrillator 1, the expiration information of the first disposable pad set 90 may be updated on the basis of the information indicated by the identification code 95. That is, the expiration information of the first disposable pad set 90 may be updated from the expiration information that is based on the identification code 95 of the second disposable pad set 90 to the expiration information that is based on the identification code 95 of the first disposable pad set 90.

Alternatively, a user may be allowed to input, to the semiautomated defibrillator 1, information indicating whether expiration information acquired by the acquisition unit 11 is based on the identification code 95 of the second disposable pad set 90 that is connected to the semiautomated defibrillator 1 or based on the identification code 95 of the first disposable pad set 90 to be connected to the semiautomated defibrillator 1 next. The information indicating which identification code 95 expiration information acquired by the acquisition unit 11 is based on can be transmitted to the computer 2 by the transmission/reception unit 13.

Where there exists, in addition to the first disposable pad set 90, near the semiautomated defibrillator 1, a disposable pad set 90 that will be connected to the semiautomated defibrillator 1 in the future, by causing the acquisition unit 11 to also acquire the expiration information of that disposable pad set 90 a managing unit 211 (described later) may manage expiration management operations for the plural respective disposable pad sets 90 in parallel. In this case, it is preferable that the identification code 95 of each disposable pad set 90 include identification information that enables identification of the disposable pad set 90.

Where the connector 91 has the holding unit and the wrapping member 94 has the identification code 95, first, the acquisition unit 11 may read the expiration information of the second disposable pad set 90 from the holding unit of the second disposable pad set 90 and acquire the thus-read expiration information as tentative expiration information of the first disposable pad set 90 to be connected to the semiautomated defibrillator 1 next. Then the acquisition unit 11 acquires the expiration information read from the identification code 95 of the first disposable pad set 90 and updates the expiration information of the first disposable pad set 90 to the acquired expiration information. If the identification code 95 is not read, expiration date management is performed on the basis of the tentative expiration information.

The transmission/reception unit 13 transmits and receives information to and from the computer 2 and the user terminal 3 via the communication unit 80. For example, the transmission/reception unit 13 transmits expiration information acquired by the acquisition unit 11 to the computer 2.

When a disposable pad set 90 is connected to the semiautomated defibrillator 1, the transmission/reception unit 13 can transmit, to the computer 2, a connection notice for notification of the connection of the disposable pad set 90. When a disposable pad set 90 is connected to the semiautomated defibrillator 1 and the semiautomated defibrillator 1 is used (i.e., an electric shock has been delivered), the transmission/reception unit 13 can transmit, to the computer 2, a use notice for notification of the use of the semiautomated defibrillator 1. Expiration information, a connection notice, and a use notice may be transmitted to the computer 2 via another device such as the user terminal 3 instead of being transmitted to the computer 2 directly.

For example, the storage unit 20 is stored with programs that are necessary for operation of the semiautomated defibrillator 1, voice data, adjustment values, electrocardiogram data of a patient, an expiration date of a disposable pad set 90 to be used next, etc. The storage unit 20 may include a secondary storage device such as a hard disk drive. Part of the storage unit 20 may be an external storage device that can be attached to and detached from the semiautomated defibrillator 1.

The manipulation receiving unit 30 receives a manipulation input from a user of the semiautomated defibrillator 1. For example, the manipulation receiving unit 30 can include a power button for starting the semiautomated defibrillator 1, a knob for adjusting the output energy, a shock button for delivering an electric shock, etc.

The display unit 40, which can include a display, displays various kinds of information on the display. For example, the various kinds of information to be displayed on the display are physiological information of a patient such as an electrocardiogram, an instruction or a message from the semiautomated defibrillator 1 to a user, etc. The display unit 40 may be equipped with a touch panel and thus function as the manipulation receiving unit 30.

The sound generation unit 50 issues various instructions to a user of the semiautomated defibrillator 1 in the form of voices by referring to the voice data stored in the storage unit 20. The high-voltage generation unit 60 performs charging and discharging to produce energy to be used for defibrillation in response to a control signal from the control unit 10. The pad connector 70 is connected to the disposable pad set 90. Energy released by the high-voltage generation unit 60 is transmitted to a patient via the pad connector 70 and the disposable pad set 90. The disposable pad set 90 takes in an electrocardiogram signal of a patient. The electrocardiogram signal is transmitted to the control unit 10 after being subjected to, for example, filtering and amplification processing. The communication unit 80 is an interface that enables a communication with the other devices.

### (Computer)

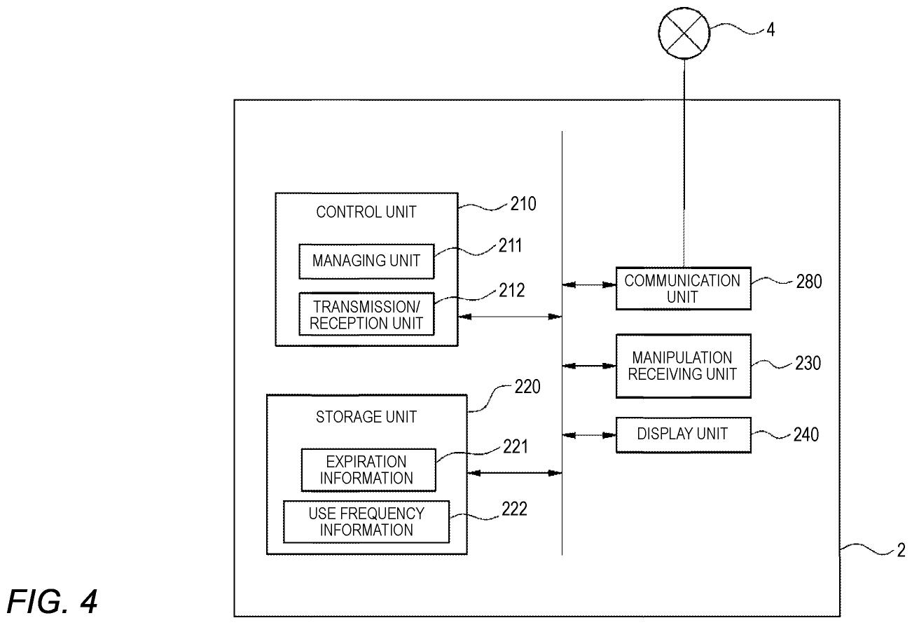

Next, the computer 2 will be described with reference to FIG. 4. The computer 2 can include a control unit 210, a storage unit 220, a manipulation receiving unit 230, a display unit 240, and a communication unit 280.

The control unit 210 can include a processor such as a CPU and memories such as a ROM and a RAM. The control unit 210 controls various operations of the computer 2. The control unit 210 also functions as a managing unit 211 and a transmission/reception unit 212.

The transmission/reception unit 212 transmits and receives information to and from the semiautomated defibrillator 1 and the user terminal 3 via the communication unit 280. For example, the transmission/reception unit 212 receives various kinds of information such as expiration information, a connection notice, and a use notice from the semiautomated defibrillator 1 or the user terminal 3.

The managing unit 211 manages the expiration date of a disposable pad set 90 to be used next on the basis of expiration information received by the transmission/reception unit 212. If the transmission/reception unit 212 has not received a connection notice a prescribed number of days before the expiration date, the managing unit 211 can output alert information. The prescribed number of days mentioned above can be set as appropriate on the basis of the types of expendable and medical device and other information. The prescribed number of days may be changeable by a manager of the computer 2 or a person belonging to the medical institution H. Example manners of output of alert information are display of the alert information on the display unit 240 or a display unit of the user terminal 3 and transmission of the alert information to the user terminal 3.

The managing unit 211 can calculate a use frequency of each semiautomated defibrillator 1 on the basis of a past use history the semiautomated defibrillator 1. More specifically, the managing unit 211 can calculate a use frequency of each semiautomated defibrillator 1 in a prescribed period on the basis of the number of times the transmission/reception unit 212 received a use notice in the prescribed period. The prescribed period mentioned above may be set as appropriate on the basis of the types of expendable and medical device and other information, and may be changeable by a manager of the computer 2 or a person belonging to the medical institution H. For example, if the transmission/reception unit 212 has received a use notice six times during the prescribed period in the case where the prescribed period is set at one year from the present date, the use frequency is “six times per year.”

If the transmission/reception unit 212 has not received a connection notice by a prescribed days before an expiration date, the managing unit 211 can determine another, recommendable semiautomated defibrillator 1 as a movement destination of the disposable pad set 90 on the basis of the use frequency and output information relating to the thus-determined semiautomated defibrillator 1 as recommendable information. Example output forms of recommendable information are display of the recommendable information on the display device 240 or the display unit of the user terminal 3 and transmission of the recommendable information to the user terminal 3.

The storage unit 220 stores programs and data that are necessary for various operations of the computer 2. In the embodiment, the storage unit 220 stores expiration information 221 and use frequency information 222. The expiration information 221 includes pieces of expiration information of plural semiautomated defibrillators 1 received by the transmission/reception unit 212. The use frequency information 222 includes pieces of information relating to use frequencies of the plural semiautomated defibrillators 1 calculated by the managing unit 211.

The manipulation receiving unit 230 receives a manipulation input from a user of the computer 2. The manipulation receiving unit 230 can be made up of physical buttons, a touch panel, or the like. The display unit 240 can be a display that displays various kinds of information. The display unit 240 may be a touch panel and may be one that also functions as the manipulation receiving unit 230. The communication unit 280 is an interface that enables communication with the other devices.

### (Example Operation of System)

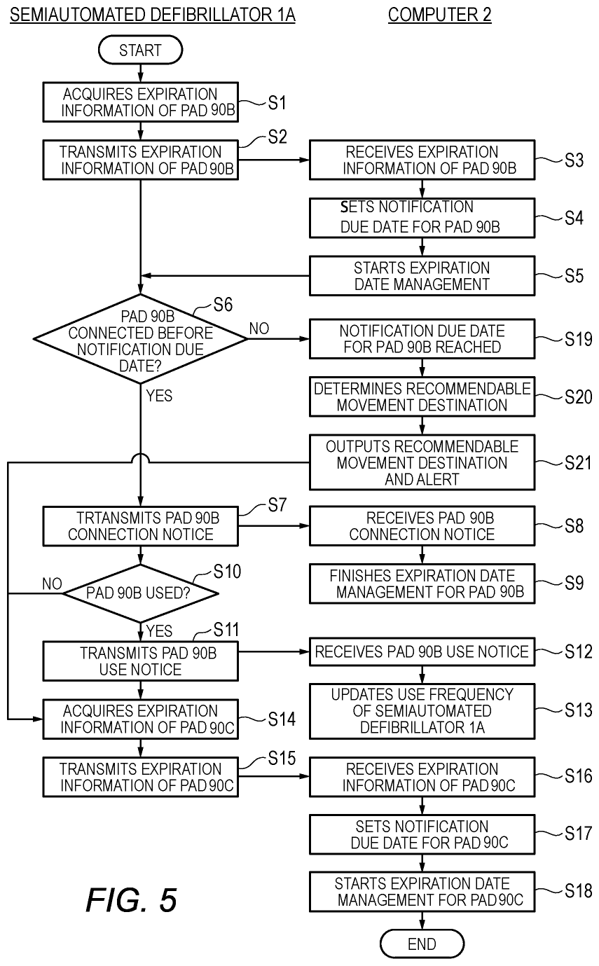

An example operation of the system 6 will be described below. FIG. 5 is a flowchart illustrating an example process that is executed by the system 6. FIG. 5 illustrates a process that is executed by the semiautomated defibrillator 1A and the computer 2, and a similar process is executed by another semiautomated defibrillator 1 such as the semiautomated defibrillator 1B and the computer 2. The order or execution of the steps constituting the flowchart may be changed within the confines that no discrepancy or mismatching occurs in the process details and part of the steps may be executed in parallel.

FIG. 5 illustrates an example process of a case that a disposable pad set 90A is already being used, a disposable pad set 90B is to be used next, and a disposable pad set 90C is to be used next to the disposable pad set 90B. First, the semiautomated defibrillator 1A acquires expiration information of the disposable pad set 90B to be connected next (step S1). Expiration information is acquired by the above-described method. Then the semiautomated defibrillator 1A transmits the expiration information acquired at step S1 to the computer 2 (step S2).

The computer 2 receives the expiration information transmitted at step S2 (step S3). Then the computer 2 sets, as a notification due date for the disposable pad set 90B, a date a prescribed number of days before the expiration date indicated by the expiration information received at step S3 (step S4). The computer 2 thereafter starts expiration date management for the disposable pad set 90B on the basis of the notification due date that was set at step S4 (step S5). At step S5, for example, the expiration date management is performed by decreasing the number of remaining days to the notification due date as time elapses.

If the disposable pad set 90B is connected to the semiautomated defibrillator 1A before the notification due date for it (step S6: yes), the process moves to step S7. The semiautomated defibrillator 1A transmits a pad 90B connection notice to the computer 2 in response to the connection of the disposable pad set 90B to the semiautomated defibrillator 1A (step S7).

After the execution of step S7, the computer 2 receives the use notice transmitted at step S7 (step S8). Then the computer 2 finishes the expiration date management for the disposable pad set 90B that was scheduled to be connected next (judged connected to the semiautomated defibrillator 1A at step S6) (step S9).

On the other hand, the semiautomated defibrillator 1A moves to step S10 after the execution of step S7. If a disposable pad set 90B was used for delivering an electric shock (step S10: yes), the semiautomated defibrillator 1A transmits a disposable pad set B use notice to the computer 2 (step S11).

The computer 2 receives the use notice transmitted at step S11 (step S12). Then the computer 2 calculates a use frequency of the semiautomated defibrillator 1A in a prescribed period on the basis of the use notice received at step S12 and updates use frequency information 222 (step S13).

The use frequency information 222 can also be updated as time elapses. For example, if the time elapsed without reception of a use notice in a state that the use frequency is six times per year and the number of times of reception of a use notice in a past one-year period before the date concerned has become five, the use frequency is updated to “five times per year.”

On the other hand, if the process moves to step S14 after the execution of step S11 or if the disposable pad set 90B was not used for delivering an electric shock (step S10: no), the semiautomated defibrillator 1A moves to step S14. The case that the disposable pad set 90B was not used for delivering an electric shock means a case that a judgment to the effect that no electric shock is necessary on the basis of, for example, an electrocardiogram of a patient. Even in this case, the disposable pad set 90B connected to the semiautomated defibrillator 1A is discarded as being use-completed.

The semiautomated defibrillator 1A acquires, as new expiration information, expiration information of a disposable pad set 90C to be used next to the disposable pad set 90B that was judged connected to the semiautomated defibrillator 1A at step S6 (step S14). Then the semiautomated defibrillator 1A transmits the new expiration information acquired at step S14 to the computer 2 (step S15).

The computer 2 receives the new expiration information transmitted at step S15 (step S16). Then the computer 2 sets, as a notification due date for the disposable pad set 90C, a date that is a prescribed number of days before the expiration date indicated by the expiration information received at step S16 (step S17). Then the computer 2 starts expiration date management for the disposable pad set 90C on the basis of the notification due date that was set at step S17 (step S18).

Next, a process that is executed when a judgment “no” is made at step S6 will be described. If the disposable pad set 90B is not connected to the semiautomated defibrillator 1A by the notification due date (step S6: no), the computer 2 recognizes that the notification due date for the disposable pad set 90B has been reached (step S19).

Subsequently, by referring to the use frequency information 222, the computer 2 determines another semiautomated defibrillator 1 that is recommended as a movement destination of the disposable pad set 90B that is the current target of the expiration date management (step S20). For example, a semiautomated defibrillator 1 that is recommended as a movement destination is a semiautomated defibrillator that is high in use frequency. The semiautomated defibrillator 1 that is recommended as a movement destination is a semiautomated defibrillator 1 (e.g., semiautomated defibrillator 1B) that is installed in the same medical institution H (e.g., same hospital).

Then the computer 2 outputs alert information relating to the disposable pad set 90B that is the current target of the expiration date management and information relating to the semiautomated defibrillator 1 that is recommended as a movement destination of the disposable pad set 90B (step S21). Step S21 can be executed repeatedly at a prescribed interval (e.g., every day) until reception of a use notice of the disposable pad set 90B that is the current target of the expiration date management or reception of information indicating that the disposable pad set 90B has been moved to the other semiautomated defibrillator 1.

If the target of the expiration date management is changed after the execution of step S21 because of the use, movement, discarding, or the like of the disposable pad set 90B that has been the target of the expiration date management, at step S14 the semiautomated defibrillator 1A acquires the expiration information of the disposable pad set 90C to be used next.

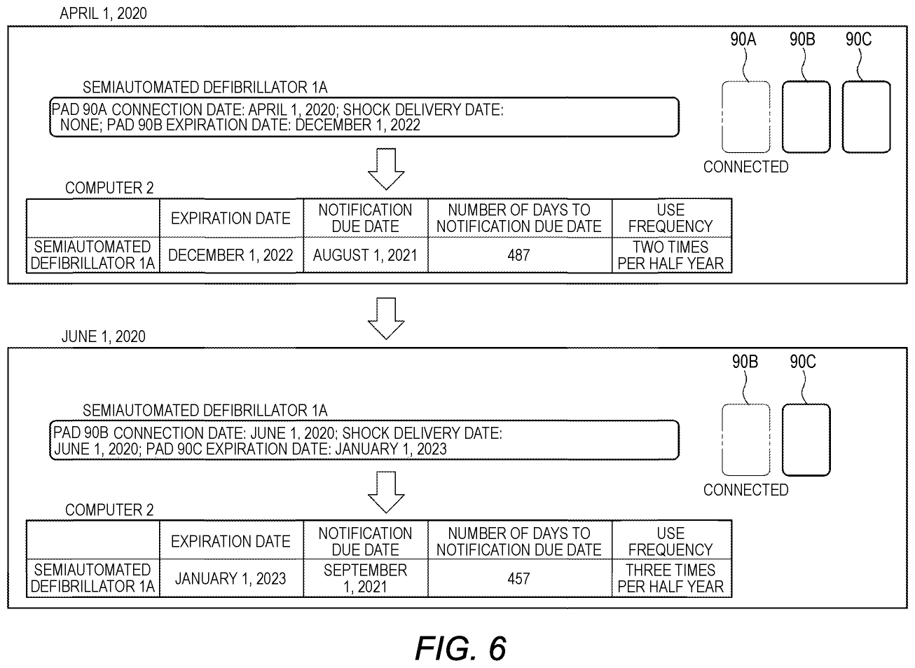

Next, the above-described process will be described in more detail with reference to FIG. 6. FIG. 6 is a diagram illustrating a specific example of the expiration date management according to the embodiment. In the example shown in FIG. 6, the number of days to the expiration date increases in order of a disposable pad set 90A, a disposable pad set 9B, and a disposable pad set 90C and the expiration date management is performed so that the disposable pad sets 90A, 90B, and 90C are connected to the semiautomated defibrillator 1A in this order.

In the example shown in FIG. 6, the disposable pad set 90A is connected to the semiautomated defibrillator 1A on Apr. 1, 2020. No electric shock is delivered on this day. As at step S1 shown in FIG. 5, the semiautomated defibrillator 1A acquires a date Dec. 1, 2022 as an expiration date of the disposable pad set 90B to be connected next.

Where the system is configured so that an expiration date is acquired from the holding unit, what the semiautomated defibrillator 1A acquires as an expiration date of the disposable pad set 90B is the expiration date of the disposable pad set 90A acquired from the holding unit of the disposable pad set 90A. That is, in this case, the expiration date held by the holding unit of the disposable pad set 90A is “Dec. 1, 2022.”

On the other hand, where the system is configured so that an expiration date is acquired from the identification code 95, what the semiautomated defibrillator 1A acquires as an expiration date of the disposable pad set 90B is the expiration date acquired from the identification code 95 that is attached to the wrapping member 94 of the disposable pad set 90B. That is, in this case, the expiration date indicated by the identification code 95 of the disposable pad set 90B is “Dec. 1, 2022.” It is noted that this example is such that the disposable pad set 90B is located near the semiautomated defibrillator 1A when the disposable pad set 90A is in use. If the disposable pad set 90B is not located near the semiautomated defibrillator 1A when the disposable pad set 90A is in use, the expiration date acquired from the identification code 95 of the disposable pad set 90A can be employed as an expiration date of the disposable pad set 90B.

The computer 2 acquires a disposable pad set 90A connection notice and the expiration information of the disposable pad set 90B from the semiautomated defibrillator 1A. Subsequently, as at step S5 shown in FIG. 5, the computer 2 starts expiration date management for the disposable pad set 90B. In the example shown in FIG. 6, a notification due date is set to a date that is one year four months before the expiration date. Thus, the notification due date is “Aug. 1, 2021” and the number of days remaining until the notification due date is 487 days. The number of remaining days is decreased as time elapses.

In the example shown in FIG. 6, the use frequency is the number of times of reception of a use notice (the number of times of use of the semiautomated defibrillator 1A) in a period from six months ago to the present date. As of Apr. 1, 2020, the use frequency is “two times per half year.” If an electric shock were delivered on Apr. 1, 2020, the use frequency would be updated to “three times per half year.” The use frequency can be updated as time elapses.

The disposable pad set 90B is connected to the semiautomated defibrillator 1A on Jun. 1, 2020. An electric shock is delivered on this day. As at step S14 shown in FIG. 5, the semiautomated defibrillator 1A acquires a date Jan. 1, 2023 as an expiration date of the disposable pad set 90C to be connected next.

As in the case of the disposable pad set 90B, what the semiautomated defibrillator 1A acquires as an expiration date of the disposable pad set 90C is, for example, the expiration date acquired from the holding unit of the disposable pad set 90B or the expiration date indicated by the identification code 95 that is attached to the disposable pad set 90C.

The computer 2 acquires a disposable pad set B connection notice and use notice and the expiration information of the disposable pad set 90C from the semiautomated defibrillator 1A. Subsequently, as at step S18 shown in FIG. 5, the computer 2 starts expiration date management for the disposable pad set 90C. Furthermore, the computer 2 updates the use frequency. A notification due date, the number of days remaining until the notification due date, and a use frequency as of Jun. 1, 2020 are as shown in FIG. 6.

If the computer 2 has not received a disposable pad set 90C use notice by the notification due date, as at step S21 shown in FIG. 5, the computer 2 outputs alert information. Although not shown in FIG. 6, the computer 2 is doing expiration date management operations for semiautomated defibrillators 1 (e.g., semiautomated defibrillator 1B) other than the semiautomated defibrillator 1A and is stored with use frequencies of the other semiautomated defibrillators 1. For example, the computer 2 decides on a semiautomated defibrillator 1 that is high in use frequency as a movement destination of the disposable pad set 90C and outputs information relating to that semiautomated defibrillator 1.

Next, advantages obtained by the system 6 according to the embodiment or the individual devices, members, etc. included in the system 6 will be described.

Each semiautomated defibrillator 1 acquires expiration information relating to a expiration date of a first disposable pad set 90 in a state that the semiautomated defibrillator 1 is not connected to the first disposable pad set 90 and transmits the acquired expiration information. The computer 2 receives that expiration information and manages the expiration date of the first disposable pad set 90 on the basis of the received expiration information. With this configuration, the expiration date of the first disposable pad set 90 can be managed remotely, that is, even if it is not connected to the semiautomated defibrillator 1.

Each disposable pad set 90 includes the holding unit which holds expiration information. When a second disposable pad set 90 is connected to the semiautomated defibrillator 1, the semiautomated defibrillator 1 reads the expiration information from the holding unit of the second disposable pad set 90 and acquires the read-out expiration information as expiration information of the first disposable pad set 90 to be connected to the semiautomated defibrillator 1 next to the second disposable pad set 90. With this configuration, the expiration date of a disposable pad set 90 to be used next can be managed so that it does not lapse even if the disposable pad set 90 is not connected to the semiautomated defibrillator 1. Furthermore, since the expiration information of the disposable pad set 90 can be acquired upon a start of its use, a failure to acquire the expiration information is not prone to occur.

Since the identification code 95 indicating expiration information is attached to a disposable pad set 90 or an attachment to it and the acquisition unit 11 acquires, as expiration information, information indicated by the identification code 95, the expiration date of the disposable pad set 90 can be acquired easily in a simple manner. Furthermore, since the expiration date of the disposable pad set 90 itself can be acquired, correct expiration date management can be performed.

When the identification code 95 attached to a second disposable pad set 90 or an attachment to it is read out, information indicated by the read-out identification code 95 is acquired as expiration information of a first disposable pad set 90 to be connected to the semiautomated defibrillator 1 next to the second disposable pad set 90. This makes it possible to manage the expiration date of the first disposable pad set 90 remotely, that is, even if the first disposable pad set 90 is not located near the semiautomated defibrillator 1.

If the transmission/reception unit 212 does not receive a connection notice by a prescribed number of days before an expiration date, the managing unit 211 outputs alert information. This makes it possible to inform a manager of the computer 2 or a user of the semiautomated defibrillator 1 that the expiration date of, for example, a disposable pad set 90 is imminent and hence to lower the probability that the disposable pad set 90 whose expiration date has lapsed is used.

The managing unit 211 calculates a use frequency of each semiautomated defibrillator 1 in a prescribed period on the basis of the number of times the transmission/reception unit 212 has received a use notice in a prescribed period. This makes it possible to recognize a use frequency of each semiautomated defibrillator 1 and manage, more properly, the semiautomated defibrillator 1 remotely.

If the transmission/reception unit 212 does not receive a connection notice by a prescribed number of days before an expiration date, the managing unit 211 determines another semiautomated defibrillator 1 as a recommendable movement destination of the disposable pad set 90 on the basis of use frequencies and outputs information relating to the other semiautomated defibrillator 1. When the expiration date of a disposable pad set 90 is imminent, this measure makes it possible to, for example, move that disposable pad set 90 to another semiautomated defibrillator that is high in use frequency. As a result, this measure can lower the probability that a disposable pad set 90 is rendered unusable because of a lapse of its expiration date.

The above embodiment is just an example for facilitating the understanding of the presently disclosed subject matter. The configurations relating to the above embodiment can be modified or improved as appropriate without departing from the spirit and scope of the presently disclosed subject matter.

The configurations disclosed above enable, in a medical device using an expendable having an expiration date as in semiautomated defibrillators, remote management of an expiration date of the expendable even if the expendable is not connected to the medical device.

What is claimed is:

1. A system comprising:
a medical device that uses a plurality of expendables each having an expiration date; and
a computer that can be connected for communication to the medical device, wherein:
the medical device includes:
an acquisition unit configured to acquire expiration information relating to an expiration date of a first expendable included in the plurality of expendables in a state that the medical device is not connected to the first expendable; and
a transmission unit configured to transmit the expiration information acquired by the acquisition unit; and
the computer includes:
a reception unit configured to receive the expiration information; and
a managing unit configured to manage the expiration date of the first expendable on the basis of the expiration information received by the reception unit.

2. The system according to claim 1, wherein:
a second extendable included in the plurality of expendables includes a holding unit configured to hold an expiration information relating to an expiration date of the second extendable;
the expiration information held by the holding unit can be read by the acquisition unit in a state that the expendable is connected to the medical device; and
when the second expendable is connected to the medical device, the acquisition unit reads expiration information from the holding unit of the second expendable and acquires the read-out expiration information as expiration information of the first expendable to be connected to the medical device next to the second expendable.

3. The system according to claim 1, wherein:
the plurality of expendables includes a second expendable;
an identification code indicating an expiration information relating to an expiration date is attached to at least one of the first and second expendables or an attachment to the at least one of the first and second expendables; and
the acquisition unit can read the identification code and, when reading the identification code, acquires, as the expiration information, information indicated by the identification code.

4. The system according to claim 3, wherein when reading an identification code attached to the second expendable or an attachment to the second expendable, the acquisition unit acquires information indicated by the identification code as expiration information of the first expendable to be connected to the medical device next to the second expendable.

5. The system according to claim 1, wherein:
the transmission unit further transmits a connection notice for notification of the connection of the first expendable when the first expendable is connected to the medical device; and
the managing unit outputs alert information if the reception unit has not received the connection notice by a prescribed number of days before the expiration date.

6. The system according to claim 1, wherein:
the transmission unit further transmits a use notice for notification of the use of the first expendable when the first expendable is connected to the medical device and is used; and
the managing unit further calculates a use frequency of the medical device in a prescribed period on the basis of the number of times the reception unit has received the use notice in the prescribed period.

7. The system according to claim 6, wherein:
when the first expendable is connected to the medical device, the transmission unit transmits a connection notice for notification of the connection of the first expendable; and
the managing unit further determines another medical device that is recommendable as a movement destination of the first expendable on the basis of the use frequency and outputs information relating to the other medical device if the reception unit has not received the connection notice by a prescribed number of days before the expiration date.

8. The system according to claim 1, wherein:
the medical device is a semiautomated defibrillator; and
the expendable is a disposable electrode pad set.

9. A medical device that uses a plurality of expendables each having an expiration date and can be connected for communication to a computer, comprising:
an acquisition unit configured to acquire expiration information relating to an expiration date of a first expendable included in the plurality of expendables in a state that the medical device is not connected to the first expendable; and
a transmission unit configured to transmit the expiration information acquired by the acquisition unit to the computer.

10. A computer that can be connected for communication to a medical device that uses a plurality of expendables each having an expiration date, wherein:
the medical device acquires expiration information relating to an expiration date of a first expendable included in the plurality of expendables in a state that the medical device is not connected to the first expendable and transmits the acquired expiration information to the computer; and
the computer comprises:
a reception unit configured to receive the expiration information; and
a managing unit configured to manage the expiration date of the first expendable on the basis of the expiration information received by the reception unit.

11. An expendable managing method that is performed in a system including a medical device that uses a plurality of expendables each having an expiration date and a computer that can be connected for communication to the medical device, comprising:
the steps, executed in the medical device, of:
acquiring expiration information relating to an expiration date of a first expendable included in the plurality of the expendables in a state that the medical device is not connected to the first expendable; and
transmitting the acquired expiration information; and
the steps, executed in the computer, of:
receiving the expiration information; and
managing the expiration date of the first expendable on the basis of the received expiration information.
